(19) United States
(12) Patent Application Publication
(10) Pub. No.: US 2025/0205240 A1
(43) Pub. Date: Jun. 26, 2025

(54) COMBINATIONS

(71) Applicant: Recurium IP Holdings, LLC, San Diego, CA (US)

(72) Inventors: Nathan Meade Jameson, San Diego, CA (US); Hooman Izadi, San Diego, CA (US); Petrus Rudolf de Jong, San Diego, CA (US); Fernando Donate, Santa Monica, CA (US); Laure Escoubet, San Diego, CA (US); Kevin Duane Bunker, Escondido, CA (US); Peter Qinhua Huang, San Diego, CA (US)

(21) Appl. No.: 19/079,930

(22) Filed: Mar. 14, 2025

Related U.S. Application Data
(63) Continuation of application No. PCT/US2023/074170, filed on Sep. 14, 2023.
(60) Provisional application No. 63/375,809, filed on Sep. 15, 2022.

(51) Int. Cl. A61K 31/519; A61K 31/437; A61K 31/506; A61K 45/06; A61P 35/00

(52) U.S. Cl. CPC A61K 31/519; A61K 31/437; A61K 31/506; A61K 45/06; A61P 35/00

(57) ABSTRACT

Disclosed herein are combinations of compounds for treating a disease or condition, such as cancer.

### INCORPORATION BY REFERENCE TO ANY PRIORITY APPLICATIONS

Any and all applications for which a foreign or domestic priority claim is identified in the Application Data Sheet as filed with the present application are hereby expressly incorporated by reference under 37 CFR 1.57, and Rules 4.18 and 20.6, including U.S. Provisional Application No. 63/375,809, filed Sep. 15, 2022. The present application is a continuation of PCT/US2023/074170, filed Sep. 14, 2023, which claims priority to U.S. Provisional Application No. 63/375,809, filed Sep. 15, 2022, all of which are hereby expressly incorporated herein by reference in their entireties.

### FIELD

The present application relates to the fields of chemistry, biochemistry and medicine. More particularly, disclosed herein are combination therapies, and methods of treating diseases and/or conditions with the combination therapies described herein.

### BACKGROUND

Cancers are a family of diseases that involve abnormal cell growth with the potential to invade or spread to other parts of the body. Cancer treatments today include surgery, hormone therapy, radiation, chemotherapy, immunotherapy, targeted therapy and combinations thereof. Survival rates vary by cancer type and by the stage at which the cancer is diagnosed. In 2021, roughly 1.9 million people will be diagnosed with cancer, and an estimated 600,000 people will die of cancer in the United States. Thus, there still exists a need for effective cancer treatments.

### SUMMARY

Some embodiments described herein relate to a combination of compounds that can include an effective amount of Compound (A), or a pharmaceutically acceptable salt thereof, and an effective amount of Compound (B), or a pharmaceutically acceptable salt of any of the foregoing. Other embodiments described herein relate to a combination of compounds that can include an effective amount of Compound (A), or a pharmaceutically acceptable salt thereof, an effective amount of Compound (B), or a pharmaceutically acceptable salt thereof, and an effective amount of Compound (C), or a pharmaceutically acceptable salt thereof.

Some embodiments described herein relate to the use of a combination of compounds for treating a disease or condition, wherein the combination includes an effective amount of Compound (A), or a pharmaceutically acceptable salt thereof, and an effective amount of Compound (B), or a pharmaceutically acceptable salt of thereof. Other embodiments described herein relate to the use of a combination of compounds in the manufacture of a medicament for treating a disease or condition, wherein the combination includes an effective amount of Compound (A), or a pharmaceutically acceptable salt thereof, and an effective amount of Compound (B), or a pharmaceutically acceptable salt thereof. Still other embodiments described herein relate to the use of a combination of compounds in a method for treating a disease or condition, wherein the combination includes an effective amount of Compound (A), or a pharmaceutically acceptable salt thereof, and an effective amount of Compound (B), or a pharmaceutically acceptable salt thereof.

Some embodiments described herein relate to the use of a combination of compounds for treating a disease or condition, wherein the combination includes an effective amount of Compound (A), or a pharmaceutically acceptable salt thereof, an effective amount of Compound (B), or a pharmaceutically acceptable salt of thereof, and an effective amount of Compound (C), or a pharmaceutically acceptable salt of thereof. Other embodiments described herein relate to the use of a combination of compounds in the manufacture of a medicament for treating a disease or condition, wherein the combination includes an effective amount of Compound (A), or a pharmaceutically acceptable salt thereof, an effective amount of Compound (B), or a pharmaceutically acceptable salt thereof, and an effective amount of Compound (C), or a pharmaceutically acceptable salt of thereof. Still other embodiments described herein relate to the use of a combination of compounds in a method for treating a disease or condition, wherein the combination includes an effective amount of Compound (A), or a pharmaceutically acceptable salt thereof, an effective amount of Compound (B), or a pharmaceutically acceptable salt thereof, and an effective amount of Compound (C), or a pharmaceutically acceptable salt of thereof.

In some embodiments, the disease or condition can be a cancer described herein.

### BRIEF DESCRIPTION OF THE DRAWINGS

FIG. 1 provides examples of BRAF inhibitors.

FIG. 2 provides examples of EGFR inhibitors.

FIG. 3 illustrates representative data obtained from a proliferation assay described herein using a WEE1 inhibitor (ZN-c3), encorafenib and a dual combination thereof in a HT-29 cell line. Percent inhibition is relative to baseline proliferation upon treatment with 0.1% DMSO.

FIG. 4 illustrates representative data obtained from a proliferation assay described herein using a WEE1 inhibitor (ZN-c3), encorafenib and a dual combination thereof in a LS411N cell line. Percent inhibition is relative to baseline proliferation upon treatment with 0.1% DMSO.

FIG. 5 illustrates representative data obtained from a proliferation assay described herein using a WEE1 inhibitor (ZN-c3), cetuximab, encorafenib and combinations thereof (dual and triple) in a HT-29 cell line. Percent inhibition is relative to baseline proliferation upon treatment with 0.1% DMSO.

FIG. 6 illustrates representative data obtained from a proliferation assay described herein using a WEE1 inhibitor (ZN-c3), cetuximab, encorafenib and combinations thereof (dual and triple) in a LS411N cell line. Percent inhibition is relative to baseline proliferation upon treatment with 0.1% DMSO.

FIG. 7 illustrates representative data obtained from tumor volume measurements taken during a HT-29 cell line-derived xenograft (CDX) study using a WEE1 inhibitor (ZN-c3), encorafenib, cetuximab and combinations thereof (dual and triple) in the HT-29 CDX model.

FIG. 8 illustrates representative data obtained from body weight measurements taken during the HT-29 CDX study of FIG. 7 using a WEE1 inhibitor (ZN-c3), encorafenib, cetuximab and combinations thereof (dual and triple).

FIG. 9 illustrates representative data obtained from tumor volume measurements taken during a LS411N CDX study using a WEE1 inhibitor (ZN-c3), encorafenib, cetuximab and combinations thereof (dual and triple) in the LS411N CDX model.

FIG. 10 illustrates representative data obtained from body weight measurements taken during the LS411N CDX study of FIG. 9 using a WEE1 inhibitor (ZN-c3), encorafenib, cetuximab and combinations thereof (dual and triple).

FIG. 11 illustrates representative data obtained from tumor volume measurements taken during a CRC769 patient-derived xenograft (PDX) study using a WEE1 inhibitor (ZN-c3), cetuximab, encorafenib and combinations thereof (dual and triple) in the CRC769 PDX model.

FIG. 12 illustrates representative data obtained from body weight measurements taken during the CRC769 PDX study of FIG. 11 using a WEE1 inhibitor (ZN-c3), cetuximab and combinations thereof (dual and triple).

FIG. 13 illustrates representative data obtained from tumor volume measurements taken during a CRC563 PDX study using a WEE1 inhibitor (ZN-c3), cetuximab, encorafenib and combinations thereof (dual and triple) in the CRC563 PDX model.

FIG. 14 illustrates representative data obtained from body weight measurements taken during the CRC563 PDX study of FIG. 13 using a WEE1 inhibitor (ZN-c3), cetuximab and combinations thereof (dual and triple).

FIG. 15 illustrates representative data obtained from tumor volume measurements taken during a CTG-1009 PDX study using a WEE1 inhibitor (ZN-c3), cetuximab, encorafenib and combinations thereof (dual and triple) in the CTG-1009 PDX model.

FIG. 16 illustrates representative data obtained from body weight measurements taken during the CTG-1009 PDX study of FIG. 15 using a WEE1 inhibitor (ZN-c3), cetuximab and combinations thereof (dual and triple).

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine. Those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium). Compounds described herein can also include all isotopes of atoms occurring in the intermediates or final compounds. Isotopes include those atoms having the same atomic number but different mass numbers. For example, isotopes of hydrogen include tritium and deuterium.

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

The term “antibody” (Ab) is used herein in the broadest sense and encompasses various antibody structures, including those made by the immune system or synthetic variants thereof, and including, but not limited to, monoclonal antibodies, polyclonal antibodies, multi-specific antibodies (e.g., bispecific antibodies) and antibody fragments so long as they exhibit the desired antigen-binding activity. An “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single-chain antibody molecules (e.g. scFv), and single-domain antibodies. Monoclonal antibodies are a type of synthetic antibody. In cancer treatment, monoclonal antibodies may kill cancer cells directly, they may block development of tumor blood vessels and/or they may help the immune system kill cancer cells.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds for Combination Therapy

Current first-line treatment options for BRAF V600E-mutant metastatic colorectal cancer (mCRC) is limited with chemotherapies with or without bevacizumab. After prior treatment, the BEACON study (ClinicalTrials.gov number, NCT02928224; EudraCT number, 2015-005805-35) represents the only Phase 3 trial to demonstrate a response and survival benefit for patients with BRAF V600E mutation mCRC who had received prior therapy. Despite the approval of encorafenib+cetuximab combination treatment in previously treated BRAF V600E-mutant mCRC, second-and third-line patients still remain a high unmet need. For instance, triplet combinations have been explored with BRAF-mutant mCRC, but while some improved response rates and/or disease control compared to doublet combinations, they were associated with higher toxicity. In particular, the combinations of dabrafenib+panitumumab, trametinib+panitumumab and dabrafenib+trametinib+panitumumab were explored in a recent trial, which resulted in an improved objective response rate (ORR) for the triplet therapy, but with an increase in certain adverse events, such as grade 3/4 diarrhea, compared to the doublet combination regimens.

Combining the doublet combinations with orthogonal pathway inhibitors may allow an additive or a synergistic combination effect with a clinically meaningful response and/or durability of response, without toxicity as observed for other triplet combinations, such as those described herein. Inhibition of WEE1 with ZN-c3 (Compound (A)), or a pharmaceutically acceptable thereof, is an attractive choice, given that WEE1 targeting is particularly effective in cancer cells with oncogene-driven replication stress, such as occurs in RAS/RAF mutant or MYC amplified cells. In line with this, some embodiments disclosed herein relate to use of a combination of compounds for treating a disease or condition, wherein the combination can include an effective amount of Compound (A), or a pharmaceutically acceptable salt thereof; and an effective amount of Compound (B), or a pharmaceutically acceptable salt thereof; wherein Compound (A) is

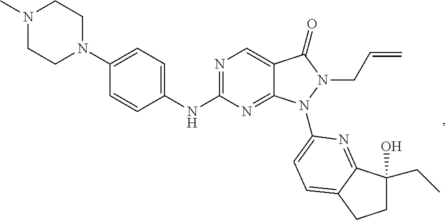 [SMILES: C=CCn1c(=O)c2cnc(Nc3ccc(N4CCN(C)CC4)cc3)nc2n1-c1ccc2c(n1)[C@@](O)(CC)CC2]

or a pharmaceutically acceptable thereof; and Compound (B) is a BRAF inhibitor, or a pharmaceutically acceptable thereof.

Some embodiments disclosed herein relate to a combination of compounds for use in treating a disease or condition, wherein the combination can include an effective amount of Compound (A), or a pharmaceutically acceptable salt thereof; and an effective amount of Compound (B), or a pharmaceutically acceptable salt thereof; wherein Compound (A) is

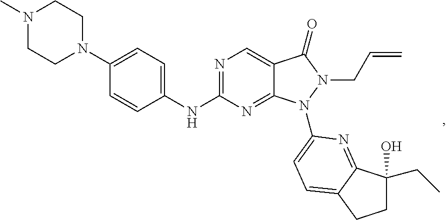 [SMILES: C=CCn1c(=O)c2cnc(Nc3ccc(N4CCN(C)CC4)cc3)nc2n1-c1ccc2c(n1)[C@@](O)(CC)CC2]

or a pharmaceutically acceptable thereof; and Compound (B) is a BRAF inhibitor, or a pharmaceutically acceptable thereof.

Compound (A), (R)-2-allyl-1-(7-ethyl-7-hydroxy-6,7-dihydro-5H-cyclopenta[b]pyridin-2-yl)-6-((4-(4-methylpiperazin-1-yl)phenyl)amino)-1,2-dihydro-3H-pyrazolo[3,4-d]pyrimidin-3-one, along with its pharmaceutically acceptable salts, can be prepared following the procedures provided in WO 2019/173082. As provided in WO 2019/173082, Compound (A) (including pharmaceutically acceptable salts thereof) is active against WEE1.

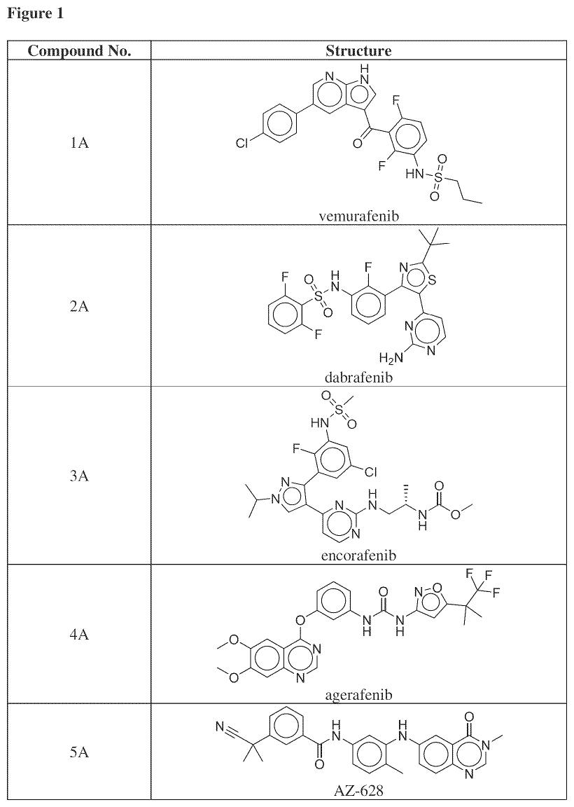

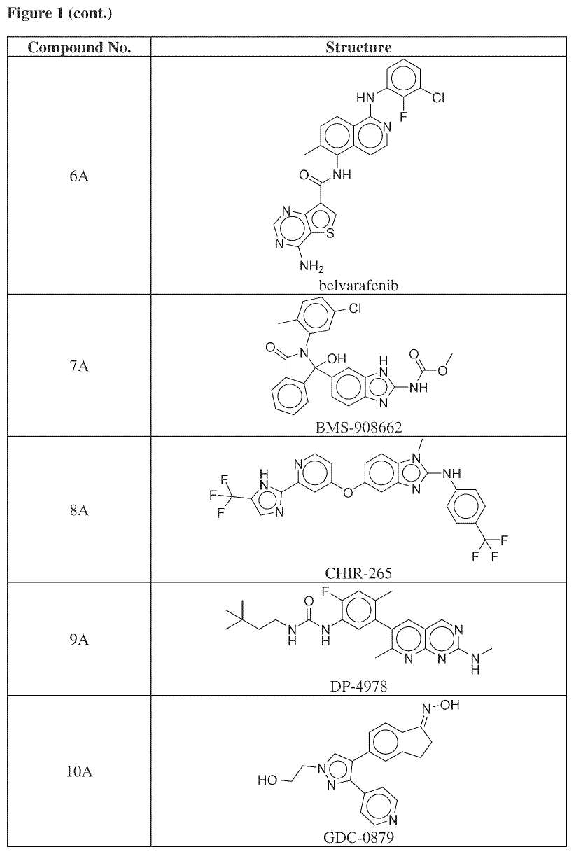

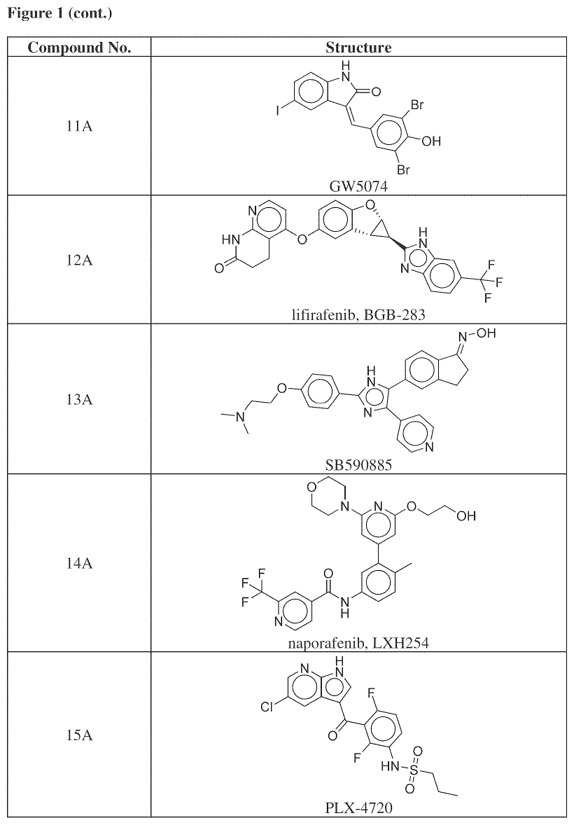

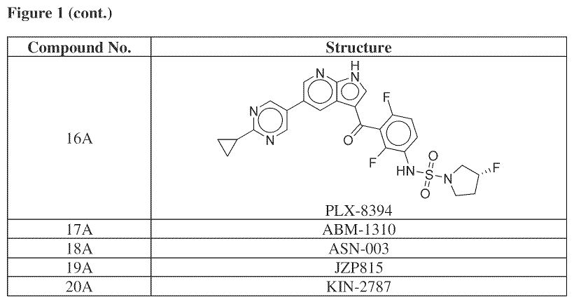

Examples of BRAF inhibitors include vemurafenib, dabrafenib, encorafenib, agerafenib, AZ-628, belvarafenib, BMS-908662, CHIR-265, DP-4978, GDC-0879, GW5074, lifirafenib, SB590885, naporafenib, PLX-4720, PLX-8394, ABM-1310, ASN-003, JZP815 and KIN-2787, or a pharmaceutically acceptable salt of any of the foregoing. FIG. 1 provides further information regarding BRAF inhibitors.

A combination described herein can further include Compound (C), including pharmaceutically acceptable salts thereof, wherein Compound (C) can be an EGFR inhibitor, or a pharmaceutically acceptable salt thereof. Accordingly, some embodiments disclosed herein relate to the use of a combination of compounds for treating a disease or condition, wherein the combination can include an effective amount of Compound (A), or a pharmaceutically acceptable salt thereof; an effective amount of Compound (B), or a pharmaceutically acceptable salt thereof; and an effective amount of Compound (C), or a pharmaceutically acceptable salt thereof; wherein Compound (A) is

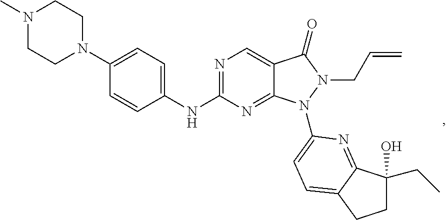 [SMILES: C=CCn1c(=O)c2cnc(Nc3ccc(N4CCN(C)CC4)cc3)nc2n1-c1ccc2c(n1)[C@@](O)(CC)CC2]

or a pharmaceutically acceptable salt thereof; Compound (B) is a BRAF inhibitor, or a pharmaceutically acceptable salt thereof; and Compound (C) is an EGFR inhibitor, or a pharmaceutically acceptable salt thereof. Furthermore, some embodiments disclosed herein relate to a combination of compounds for use in treating a disease or condition, wherein the combination can include an effective amount of Compound (A), or a pharmaceutically acceptable salt thereof; an effective amount of Compound (B), or a pharmaceutically acceptable salt thereof; and an effective amount of Compound (C), or a pharmaceutically acceptable salt thereof; wherein Compound (A) is

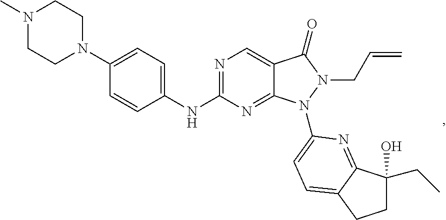 [SMILES: C=CCn1c(=O)c2cnc(Nc3ccc(N4CCN(C)CC4)cc3)nc2n1-c1ccc2c(n1)[C@@](O)(CC)CC2]

or a pharmaceutically acceptable salt thereof; Compound (B) is a BRAF inhibitor, or a pharmaceutically acceptable salt thereof; and Compound (C) is an EGFR inhibitor, or a pharmaceutically acceptable salt thereof.

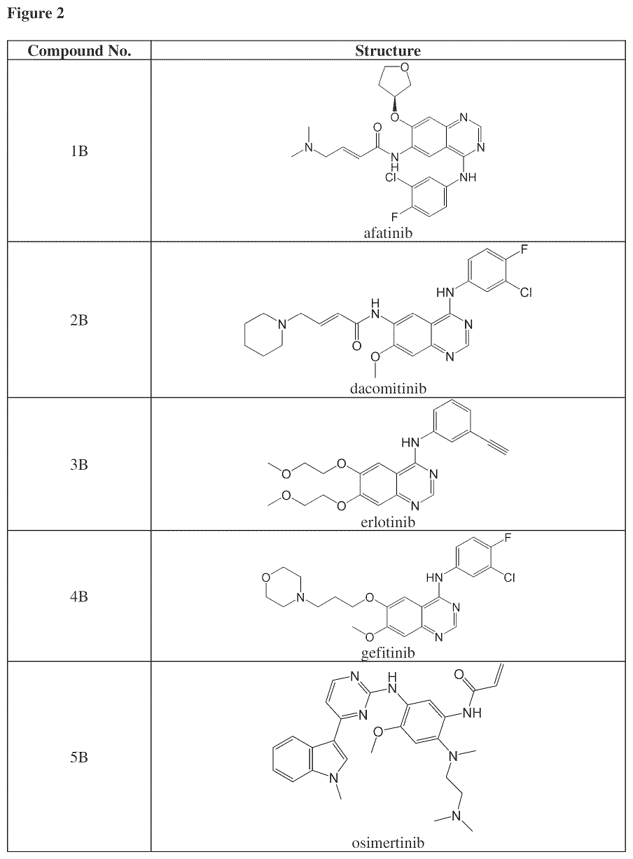

In some embodiments, the EGFR inhibitor can be a tyrosine kinase inhibitor (TKI). In other embodiments, the EGFR inhibitor can be an antibody, such as, but not limited to, a monoclonal antibody, or an antigen-binding fragment thereof. Examples of EGFR inhibitors include afatinib, dacomitinib, erlotinib, gefitinib, osimertinib, cetuximab, necitumumab, nimotuzumab, panitumumab and N-(5-((4-(1-(bicyclo[1.1.1]pentan-1-yl)-1H-indol-3-yl)pyrimidin-2-yl)amino)-2-((2-(dimethylamino)ethyl)(methyl)amino)-4-methoxyphenyl)acrylamide (along with pharmaceutically acceptable salts thereof). FIG. 2 provides further information regarding EGFR inhibitors.

Embodiments of combinations of Compound (A) and Compound (B), including pharmaceutically acceptable salts of any of the foregoing, and embodiments of Compound (A), Compound (B) and Compound (C), including pharmaceutically acceptable salts of any of the foregoing, are provided in Table 1. In Table 1, “A” indicates Compound (A) (including pharmaceutically acceptable salts thereof), numbers 1A-20A correspond to Compound (B) (including pharmaceutically acceptable salts thereof) provided in FIG. 1, and the numbers 1B-10B correspond to Compound (C) (including pharmaceutically acceptable salts thereof) provided in FIG. 2, including pharmaceutically acceptable salts thereof.

TABLE 1
Cmpd:Cmpd

A:1A
A:2A
A:3A
A:4A
A:5A
A:6A
A:7A
A:8A
A:9A
A:10A
A:1A:1B
A:2A:1B
A:3A:1B
A:4A:1B
A:5A:1B
A:6A:1B
A:7A:1B
A:8A:1B
A:9A:1B
A:10A:1B
A:1A:1C
A:2A:2B
A:3A:2B
A:4A:2B
A:5A:2B
A:6A:2B
A:7A:2B
A:8A:2B
A:9A:2B
A:10A:2B
A:1A:3B
A:2A:3B
A:3A:3B
A:4A:3B
A:5A:3B
A:6A:3B
A:7A:3B
A:8A:3B
A:9A:3B
A:10A:3B
A:1A:4B
A:2A:4B
A:3A:4B
A:4A:4B
A:5A:4B
A:6A:4B
A:7A:4B
A:8A:4B
A:9A:4B
A:10A:4B
A:1A:5B
A:2A:5B
A:3A:5B
A:4A:5B
A:5A:5B
A:6A:5B
A:7A:5B
A:8A:5B
A:9A:5B
A:10A:5B
A:1A:6B
A:2A:6B
A:3A:6B
A:4A:6B
A:5A:6B
A:6A:6B
A:7A:6B
A:8A:6B
A:9A:6B
A:10A:6B
A:1A:7B
A:2A:7B
A:3A:7B
A:4A:7B
A:5A:7B
A:6A:7B
A:7A:7B
A:8A:7B
A:9A:7B
A:10A:7B
A:1A:8B
A:2A:8B
A:3A:8B
A:4A:8B
A:5A:8B
A:6A:8B
A:7A:8B
A:8A:8B
A:9A:8B
A:10A:8B
A:1A:9B
A:2A:9B
A:3A:9B
A:4A:9B
A:5A:9B
A:6A:9B
A:7A:9B
A:8A:9B
A:9A:9B
A:10A:9B
A:1A:10B
A:2A:10B
A:3A:10B
A:4A:10B
A:5A:10B
A:6A:10B
A:7A:10B
A:8A:10B
A:9A:10B
A:10A:10B

The order of administration of compounds in a combination described herein can vary. In some embodiments, Compound (A), including pharmaceutically acceptable salts thereof, can be administered prior to Compound (B), or a pharmaceutically acceptable salt thereof. In other embodiments, Compound (A), including pharmaceutically acceptable salts thereof, can be administered concomitantly with Compound (B), or a pharmaceutically acceptable salt thereof. In still other embodiments, Compound (A), including pharmaceutically acceptable salts thereof, can be administered subsequent to the administration of Compound (B), or a pharmaceutically acceptable salt thereof. In some embodiments, when Compound (C), including pharmaceutically acceptable salts thereof, can be administered prior to both Compound (A) and Compound (B), including pharmaceutically acceptable salts of any of the foregoing. In other embodiments, when Compound (C), including pharmaceutically acceptable salts thereof, can be administered subsequent to both Compound (A) and Compound (B), including pharmaceutically acceptable salts of any of the foregoing. In still other embodiments, when Compound (C), including pharmaceutically acceptable salts thereof, can be administered prior to one of Compound (A), including pharmaceutically acceptable salts thereof, and subsequent to Compound (B), including pharmaceutically acceptable salts thereof. In yet still other embodiments, when Compound (C), including pharmaceutically acceptable salts thereof, can be administered prior to one of Compound (B), including pharmaceutically acceptable salts thereof, and subsequent to Compound (A), including pharmaceutically acceptable salts thereof.

There may be several advantages for using a combination of compounds described herein. For example, combining compounds that attack multiple pathways at the same time, can be more effective in treating a cancer, such as those described herein, compared to when the compounds of combination are used as monotherapy.

In some embodiments, a combination as described herein (such as Compound (A), including pharmaceutically acceptable salts thereof, and Compound (B), or pharmaceutically acceptable salts thereof, and Compound (A), including pharmaceutically acceptable salts thereof, Compound (B), or pharmaceutically acceptable salts thereof, and Compound (C), or pharmaceutically acceptable salts thereof), can decrease the number and/or severity of side effects that can be attributed to a compound described herein, such as Compound (B), or a pharmaceutically acceptable salt thereof.

Using a combination of compounds described herein can result in additive, synergistic or strongly synergistic effect. A combination of compounds described herein can result in an effect that is not antagonistic.

In some embodiments, a combination as described herein (such as Compound (A), including pharmaceutically acceptable salts thereof, and Compound (B), or pharmaceutically acceptable salts thereof, and Compound (A), including pharmaceutically acceptable salts thereof, Compound (B), or pharmaceutically acceptable salts thereof, and Compound (C), or pharmaceutically acceptable salts thereof), can result in an additive effect. In some embodiments, a combination as described herein (for example, Compound (A), including pharmaceutically acceptable salts thereof, and Compound (B), or pharmaceutically acceptable salts thereof, and Compound (A), including pharmaceutically acceptable salts thereof, Compound (B), or pharmaceutically acceptable salts thereof, and Compound (C), or pharmaceutically acceptable salts thereof), can result in a synergistic effect. In some embodiments, a combination as described herein (for example, Compound (A), including pharmaceutically acceptable salts thereof, and Compound (B), or pharmaceutically acceptable salts thereof, and Compound (A), including pharmaceutically acceptable salts thereof, Compound (B), or pharmaceutically acceptable salts thereof, and Compound (C), or pharmaceutically acceptable salts thereof), can result in a strongly synergistic effect. In some embodiments, a combination as described herein (such as Compound (A), including pharmaceutically acceptable salts thereof, and Compound (B), or pharmaceutically acceptable salts thereof, and Compound (A), including pharmaceutically acceptable salts thereof, Compound (B), or pharmaceutically acceptable salts thereof, and Compound (C), or pharmaceutically acceptable salts thereof), is not antagonistic.

As used herein, the term “antagonistic” means that the activity of the combination of compounds is less compared to the sum of the activities of the compounds in combination when the activity of each compound is determined individually (i.e., as a single compound). As used herein, the term “synergistic effect” means that the activity of the combination of compounds is greater than the sum of the individual activities of the compounds in the combination when the activity of each compound is determined individually. As used herein, the term “additive effect” means that the activity of the combination of compounds is about equal to the sum of the individual activities of the compounds in the combination when the activity of each compound is determined individually.

A potential advantage of utilizing a combination as described herein may be a reduction in the required amount(s) of the compound(s) that is effective in treating a disease condition disclosed herein compared to when each compound is administered as a monotherapy. For example, the amount of Compound (B), or a pharmaceutically acceptable salt thereof, used in a combination described herein can be less compared to the amount of Compound (B), or a pharmaceutically acceptable salt thereof, needed to achieve the same reduction in a disease marker (for example, tumor size) when administered as a monotherapy. Another potential advantage of utilizing a combination as described herein is that the use of two or more compounds having different mechanisms of action can create a higher barrier to the development of resistance compared to when a compound is administered as monotherapy. Additional advantages of utilizing a combination as described herein may include little to no cross resistance between the compounds of a combination described herein; different routes for elimination of the compounds of a combination described herein; and/or little to no overlapping toxicities between the compounds of a combination described herein.

### Pharmaceutical Compositions

Compound (A), including pharmaceutically acceptable salts thereof, can be provided in a pharmaceutical composition. Likewise, Compound (B) and Compound (C), including pharmaceutically acceptable salts of any of the foregoing, can be provided in a pharmaceutical composition(s).

The term “pharmaceutical composition” refers to a mixture of one or more compounds and/or salts disclosed herein with other chemical components, such as diluents, carriers and/or excipients. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal- chelating agent. A “diluent” is a type of excipient.

In some embodiments, Compound (B), along with pharmaceutically acceptable salts thereof, can be provided in a pharmaceutical composition that includes Compound (A), including pharmaceutically acceptable salts thereof. In other embodiments, Compound (B), along with pharmaceutically acceptable salts thereof, can be administered in a pharmaceutical composition that is separate from a pharmaceutical composition that includes Compound (A), including pharmaceutically acceptable salts thereof. When Compound (C), including pharmaceutically acceptable salts thereof, is included, Compound (C), including pharmaceutically acceptable salts thereof, can be provided in a pharmaceutical composition that includes Compound (A), along with pharmaceutically acceptable salts thereof, and/or Compound (B), along with pharmaceutically acceptable salts thereof. In other instances, Compound (C), including pharmaceutically acceptable salts thereof, can be provided in a separate pharmaceutical composition from Compound (A), along with pharmaceutically acceptable salts, and Compound (B), along with pharmaceutically acceptable salts.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, Compound (A), including pharmaceutically acceptable salts thereof, can be administered orally. In some embodiments, Compound (A), including pharmaceutically acceptable salts thereof, can be provided to a subject by the same route of administration as Compound (B), along with pharmaceutically acceptable salts thereof, and/or Compound (C), along with pharmaceutically acceptable salts thereof. In other embodiments, Compound (A), including pharmaceutically acceptable salts thereof, can be provided to a subject by a different route of administration as Compound (B), along with pharmaceutically acceptable salts thereof, and/or Compound (C), along with pharmaceutically acceptable salts thereof.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container, and labeled for treatment of an indicated condition.

### Uses and Methods of Treatment

As provided herein, in some embodiments, a combination of compounds that includes an effective amount of Compound (A), including pharmaceutically acceptable salts thereof, and an effective amount of Compound (B), or a pharmaceutically acceptable salt thereof, can be used to treat a disease or condition. Accordingly, some embodiments disclosed herein relate to a method of treating a disease or condition, comprising administering to a subject a combination of compounds; wherein the combination can include an effective amount of Compound (A), or a pharmaceutically acceptable salt thereof; and an effective amount of Compound (B), or a pharmaceutically acceptable salt thereof; wherein Compound (A) and Compound (B) are as defined herein. In some embodiments, a combination of compounds that includes an effective amount of Compound (A), including pharmaceutically acceptable salts thereof, an effective amount of Compound (B), including pharmaceutically acceptable salts thereof, and an effective amount of Compound (C), including pharmaceutically acceptable salts thereof, can be used to treat a disease or condition. Accordingly, some embodiments disclosed herein relate to a method of treating a disease or condition, comprising administering to a subject a combination of compounds; wherein the combination can include an effective amount of Compound (A), or a pharmaceutically acceptable salt thereof; an effective amount of Compound (B), or a pharmaceutically acceptable salt thereof; and an effective amount of Compound (C), or a pharmaceutically acceptable salt thereof; wherein Compound (A), Compound (B), and Compound (C) are as defined herein.

In some embodiments, the disease or condition can be colorectal cancer. In one embodiment, the disease or condition can be advanced colorectal cancer. In one embodiment, the disease or condition can be metastatic colorectal cancer. In one embodiment, the disease or condition can be advanced and/or metastatic colorectal cancer that has progressed following one or two prior treatment regimens, such as those described herein. BRAF mutations (i.e., mutations at the BRAF gene) can occur in colorectal cancer. For example, the BRAF mutation can be an activating mutation. In one embodiment, at least one of the BRAF mutation can be a BRAF mutation occurring at the V600 codon. In some embodiments, the BRAF mutation can be V600E, with a substitution from valine at the codon to glutamic acid. In one embodiment, the disease or condition can be BRAF V600E-mutant metastatic colorectal cancer.

In some cases, following cancer treatment, a subject can relapse or have reoccurrence of the cancer. As used herein, the terms “relapse” and “reoccurrence” are used in their normal sense as understood by those skilled in the art. Thus, the cancer can be a recurrent cancer.

As used herein, a “subject” refers to an animal that is the object of treatment, observation or experiment. “Animal” includes cold-and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant. In other embodiments, the subject can be an adult. In one embodiment, the subject can be a human with advanced colorectal cancer. In one embodiment, the subject can be a human with metastatic colorectal cancer. In one embodiment, the subject can be a human with advanced and/or metastatic colorectal cancer whose disease has progressed following one or two prior treatment regimens.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The term “effective amount” is used to indicate an amount of an active compound, or pharmaceutical agent, which elicits the biological or medicinal response indicated. For example, an effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound, or radiation, is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor.

The amount of compound, salt and/or composition required for use in treatment will vary not only with the particular compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, which is the dosage levels necessary to achieve the desired result, can be accomplished by one skilled in the art using routine methods, for example, human clinical trials, in vivo studies and in vitro studies. For example, useful dosages of Compounds (A), (B) and/or (C), or pharmaceutically acceptable salts of the foregoing, can be determined by comparing their in vitro activity, and in vivo activity in animal models. Such comparison can be done by comparison against an established drug, such as cisplatin and/or gemcitabine.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

Additional embodiments are disclosed in further detail in the following examples, which are not in any way intended to limit the scope of the claims.

### Example 1: In Vitro Tumor Cell Proliferation Assays

Cell suspensions containing 1.5×10³ total cells in 100 μL culture media were deposited in each well of an ultra-low attachment 96 well plate. Plates were incubated at 37° C. with 95% oxygen and 5% CO₂ for 72 h to allow for spheroid formation. After 72 h, 10 μL culture media with or without Cetuximab was deposited into each well to bring the final volume to 110 μL. ZN-c3 and/or Encorafenib compounds in DMSO were deposited into the plates using an automated drug dispenser at the indicated concentrations. Total DMSO content was normalized to 0.1% of the total volume in all assay conditions. Plates were sealed with breathable film to reduce evaporation and incubated at 37° C. with 95% oxygen and 5% CO2 for 192 h. After 192 h, plates were removed from the incubator and allowed to come to room temperature. 100 μL room temperature 3D CTG reagent (Promega, Cat #G9683) was added to each well. Plates were agitated at 520 revolutions per minute (rpm) for 5 mins, allowed to stabilize protected from light for 30 mins, then luminescence was measured on an M5e plate reader (SpectraMax). Percent viability was calculated as percentage of cell viability relative to DMSO-only vehicle control.

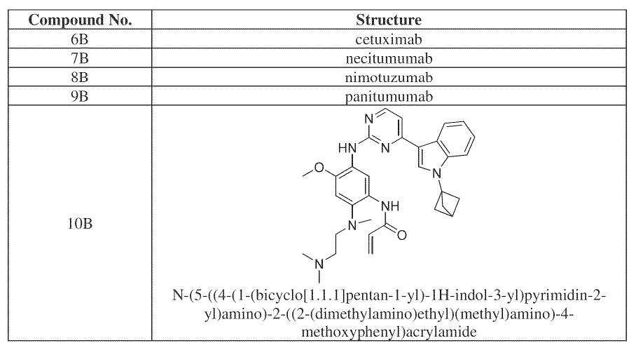

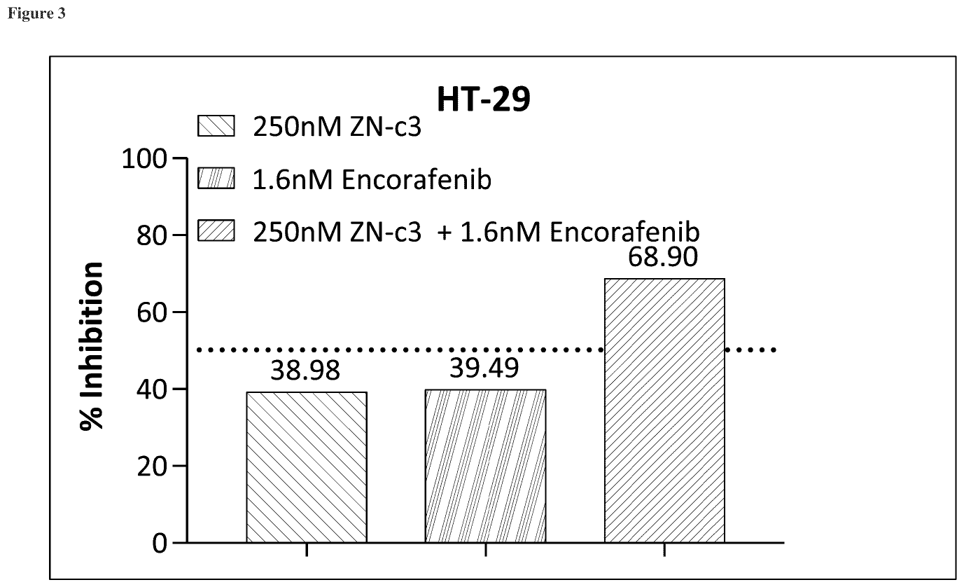

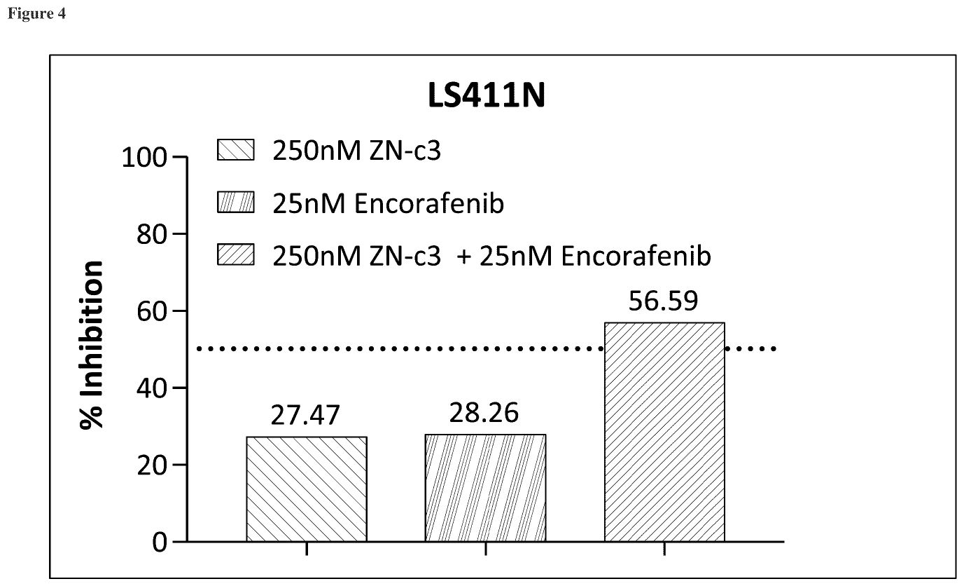

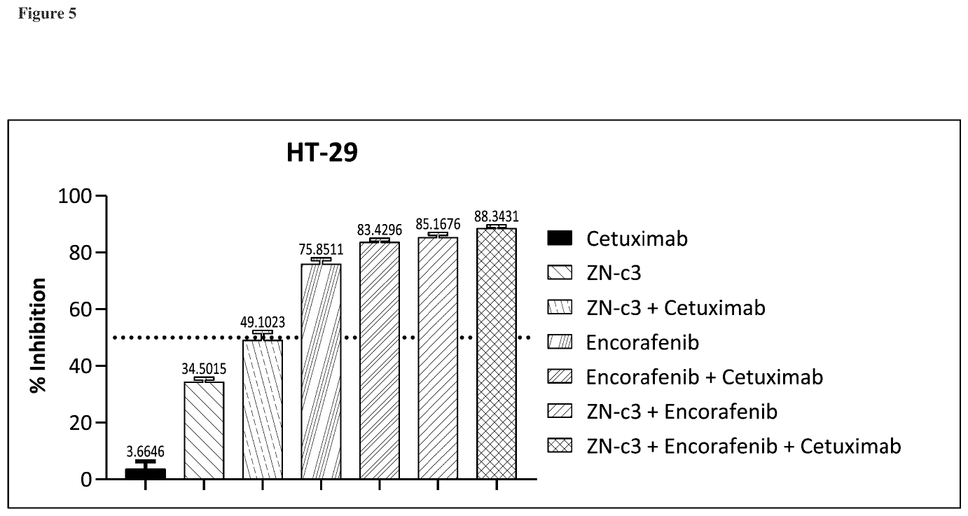

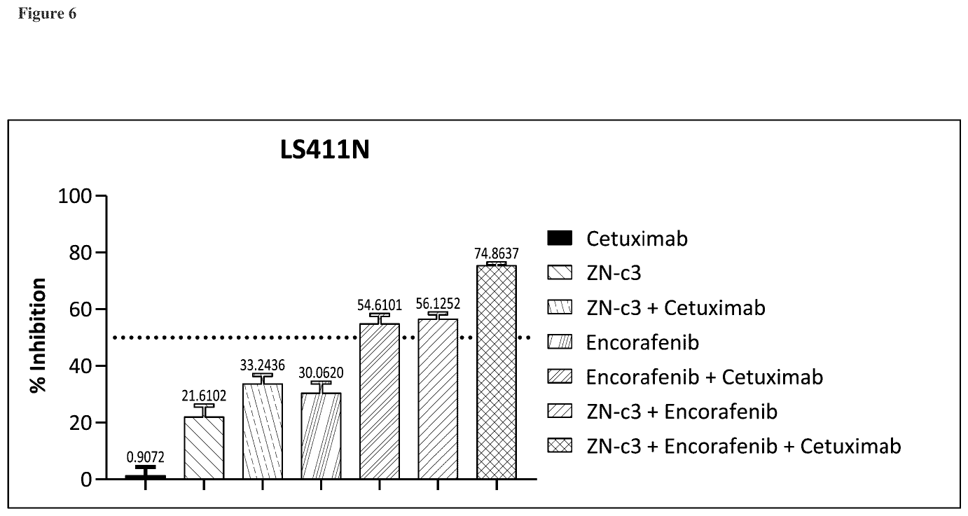

Tables 2 and 3 as well as accompanying FIGS. 3-6 detail the compounds used in the cell proliferation assays and their concentrations as well as percent inhibition of tumor cells. The percent inhibition values indicate that, with both HT-29 and LS411N cells, any double agent treatment that included ZN-c3 (i.e., ZN-c3+encorafenib or ZN-c3+cetuximab) was able to inhibit tumor cell proliferation better than any of their respective single agent treatment, e.g., ZN-c3+encorafenib inhibited tumor cell proliferation better than encorafenib alone and ZN-c3+cetuximab inhibited tumor cell proliferation better than cetuximab alone. In HT-29, double agent treatment with ZN-c3+encorafenib and triple agent treatment with ZN-c3+encorafenib+cetuximab achieved similar tumor cell inhibition as double agent treatment with encorafenib+cetuximab (See FIG. 5). In LS411N, double agent treatment with ZN-c3+encorafenib achieved similar tumor cell inhibition as double agent treatment with encorafenib+cetuximab, but triple agent treatment with ZN-c3+encorafenib+cetuximab achieved higher tumor cell inhibition than double agent treatment with encorafenib+cetuximab (See FIG. 6).

TABLE 2
HT-29

| | |
|---|---|
| Compound | % Inhibition |

ZN-c3 and encorafenib single and double agent combinations

| | |
|---|---|
| 250 nM ZN-c3 | 38.98 |
| 1.6 nM encorafenib | 39.49 |
| 250 nM ZN-c3 + | 68.90 |
| 1.6 nM encorafenib | |

ZN-c3, encorafenib and cetuximab single,
double and triple agent combinations

| | |
|---|---|
| 25 μg/mL cetuximab | 3.66 |
| 300 nM ZN-c3 | 34.50 |
| 25 μg/mL cetuximab + | 49.10 |
| 300 nM ZN-c3 | |
| 10 nM encorafenib | 75.85 |
| 10 nM encorafenib + | 83.43 |
| 25 μg/mL cetuximab | |
| 300 nM ZN-c3 + | 85.17 |
| 10 nM encorafenib | |
| 300 nM ZN-c3 + | 88.34 |
| 10 nM encorafenib + | |
| 25 μg/mL cetuximab | |

TABLE 3
LS411N

| | |
|---|---|
| Compound | % Inhibition |

ZN-c3 and encorafenib single and double agent combinations

| | |
|---|---|
| 250 nM ZN-c3 | 27.47 |
| 25 nM encorafenib | 28.26 |
| 250 nM ZN-c3 + | 56.59 |

ZN-c3, encorafenib and cetuximab single,
double and triple agent combinations

| | |
|---|---|
| 25 μg/mL cetuximab | 0.91 |
| 350 nM ZN-c3 | 21.61 |

ZN-c3, encorafenib and cetuximab single,
double and triple agent combinations

| | |
|---|---|
| 25 μg/mL cetuximab + | 33.24 |
| 350 nM ZN-c3 | |
| 75 nM encorafenib | 30.06 |
| 75 nM encorafenib + | 54.61 |
| 25 μg/mL cetuximab | |
| 350 nM ZN-c3 + | 56.13 |
| 75 nM encorafenib | |
| 350 nM ZN-c3 + | 74.86 |
| 75 nM encorafenib + | |
| 25 μg/mL cetuximab | |

### In Vivo Pre-Clinical Studies on Cell Line-Derived Xenograft (CDX) and Patient-Derived Xenograft Models

CDX Models: 6-8 week old BALB/c nude mice were inoculated subcutaneously on the right flank with the single cell suspension of 95% viable tumor cells (5×10⁶) in 100 μL RPMI 1640 without serum (COLO205), 95% viable tumor cells (2×10⁶) in 100 μL McCoy's 5a Medium Modified without serum (HT-29), or 95% viable tumor cells (2×106) in 100 μL RPMI 1640 and Matrigel mixture (1:1 ratio) without serum (LS411N).

PDX Models: Tumor fragments (CRC769, CRC563, CTG-1009) were brought out of cryopreservation and implanted into female athymic nude Foxn1nu mice. Fragments were allowed to grow then excised once an appropriate volume was reached. A tumor slurry was made of 50% freshly harvested tumors minced to small tumor fragments in PBS and 50% Matrigel. The tumor slurry was injected subcutaneously into 6-8 week old female athymic nude Foxn1nu mice on the flank.

Grouping and treatments started when the mean tumor volume reached about 220 mm³ (with individual tumors in the range of 200-240 mm³). Vehicle animals were treated daily with 10 mL/kg HP-β-CD p.o. (oral gavage). Encorafenib was prepared weekly in 0.5% carboxymethyl cellulose: 0.5% Tween 80:99% deionized water and dosed daily (QD) at the indicated doses orally (p.o.) (see Tables 4 and 5 for dosage amounts). ZN-c3 was prepared daily in 20% HP-B-CD and dosed daily at the indicated doses p.o. (see Tables 4 and 5 for dosage amounts). Cetuximab was diluted in PBS Buffer pH 7.0 at the time of dosing and dosed biweekly (BIW) at the indicated doses intraperitoneally (i.p.) (see Tables 4 and 5 for dosage amounts). Body weight and tumor volume of all animals was measured twice weekly throughout the studies that were 21 or 22 days or 3 weeks in duration (for CDX models HT-29 and LS411N) or that were 28 days or 4 weeks in duration (for PDX models CRC769, CRC563 and CTG-1009). The measurement of tumor size was performed with a caliper and the tumor volume (mm3) was estimated using the formula: TV=a×b2/2 throughout the study, where “a” and “b” is long and short diameters of a tumor, respectively. Animals were euthanized when their individual tumor burden exceeded 2000 mm³ or is in a continuing deteriorating condition or close to a comatose state.

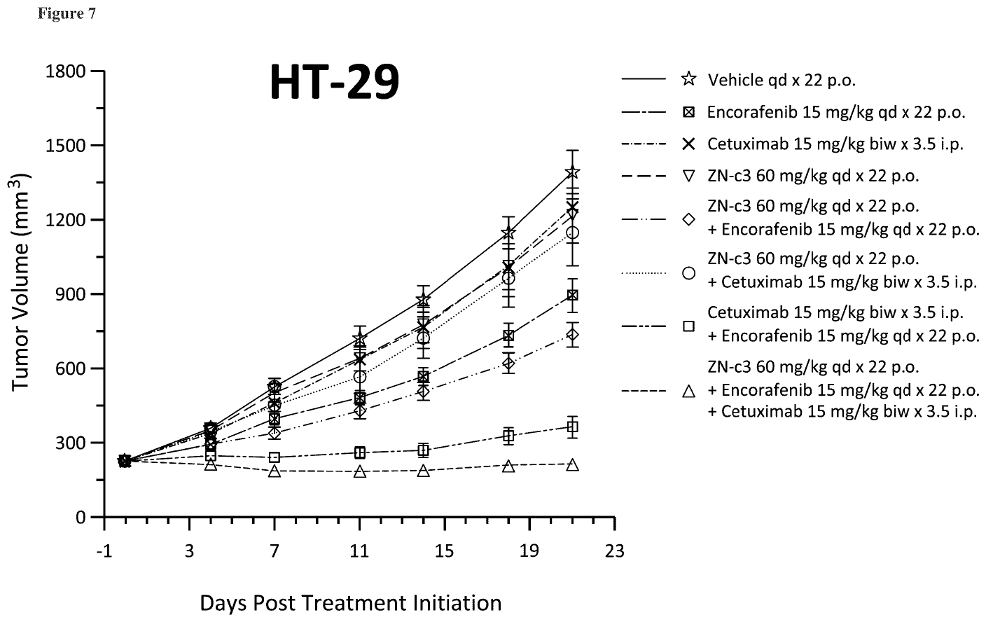

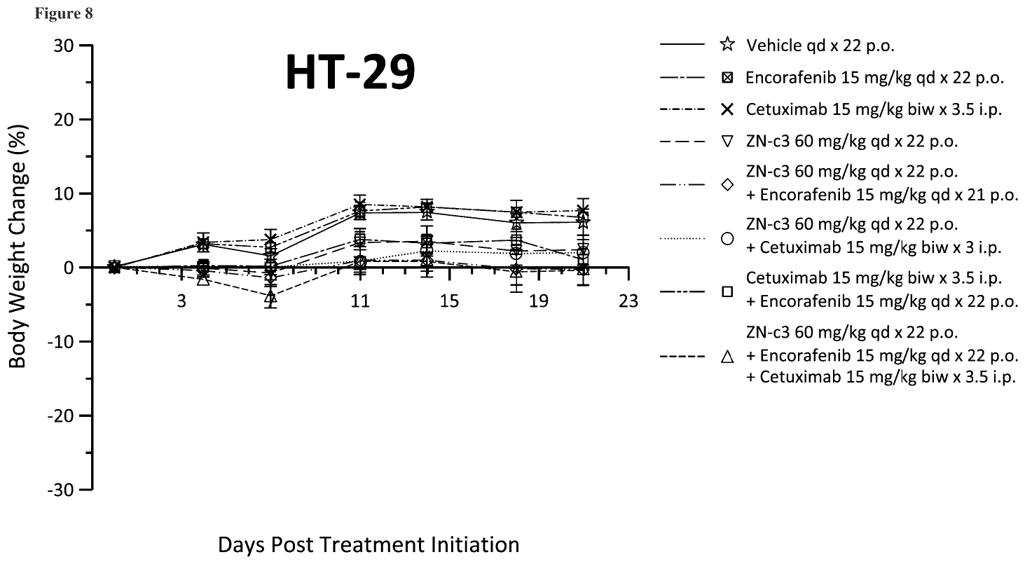

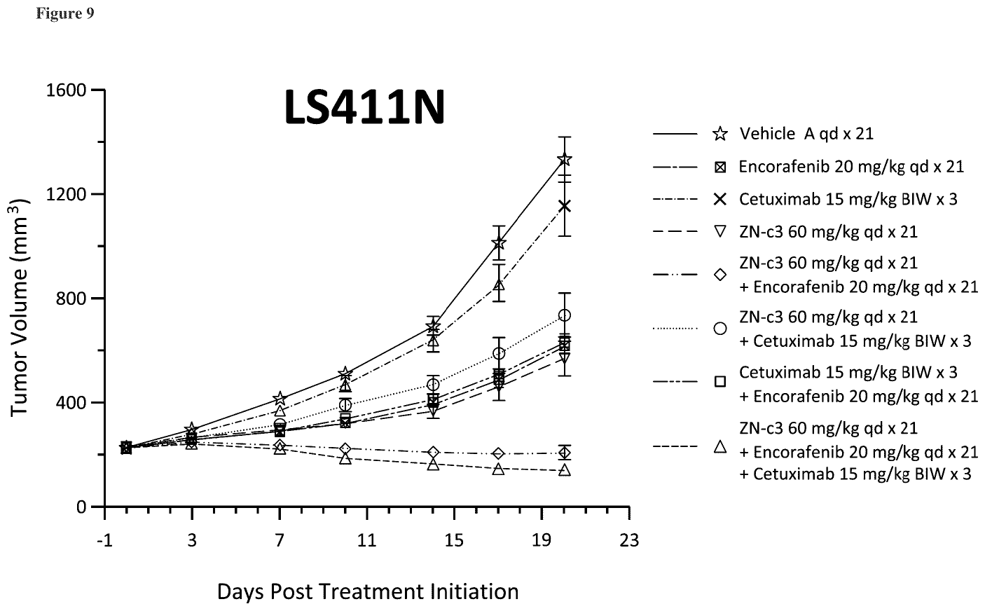

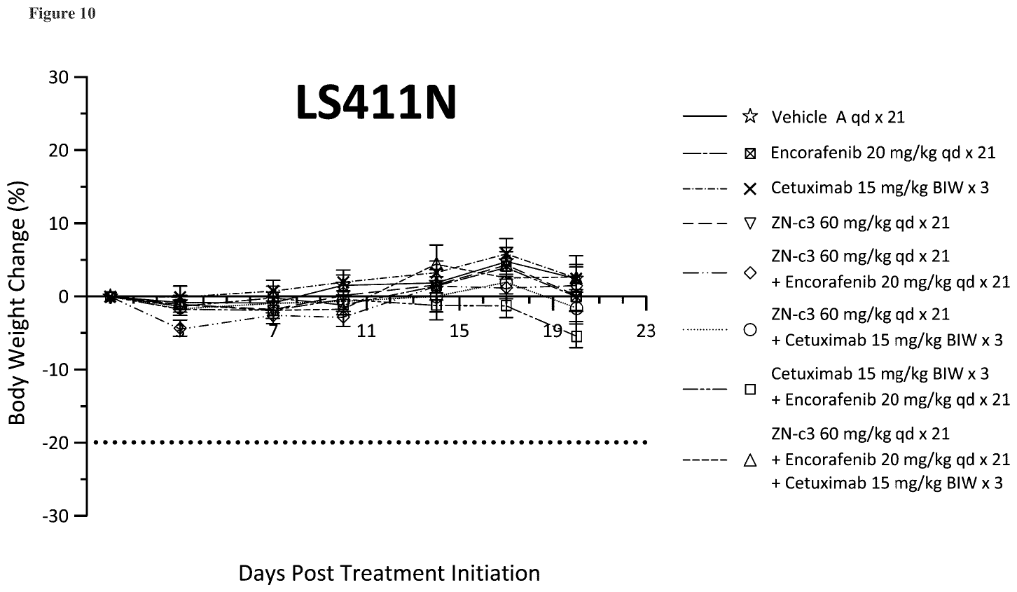

The tumor volume measurements (6 measurements altogether throughout the 21- or 22-day studies) for the CDX models are provided in FIG. 7 (HT-29 which is BRAF^{mt} CRC) and FIG. 9 (LS411N which is also BRAF^{mt} CRC). Based on the tumor volume measurements, tumor growth inhibition (TGI) values were calculated and are provided in Table 4. In both CDX models, the triplet therapy of ZN-c3+encorafenib+cetuximab induced tumor regressions that were superior to the current metastatic colorectal cancer standard-of-care double therapy of encorafenib+cetuximab, with TGI values of 100.9 (ZN-c3+encorafenib+cetuximab) as compared to 88.2 (encorafenib+cetuximab) in HT-29 and significantly, 107.6 (ZN-c3+encorafenib+cetuximab) as compared to 63.4 (encorafenib+cetuximab) in LS411N. Furthermore, in LS411N, the doublet therapy of ZN-c3+encorafenib induced tumor inhibition that were superior to the standard of care, with a TGI value of 101.7. The body weight measurements (6 measurements altogether throughout the 21- or 22-day studies) for the CDX models, as shown in FIG. 8 for HT-29 and FIG. 10 for LS411N and also in Table 4 for both models, indicated minimal body weight changes throughout the studies for the exemplary combination therapies, i.e., triplet therapy of ZN-c3+encorafenib+cetuximab and doublet therapy of encorafenib+cetuximab. Typically, a body weight loss of greater than 15% is indicative of the treatment regimen not being well-tolerated.

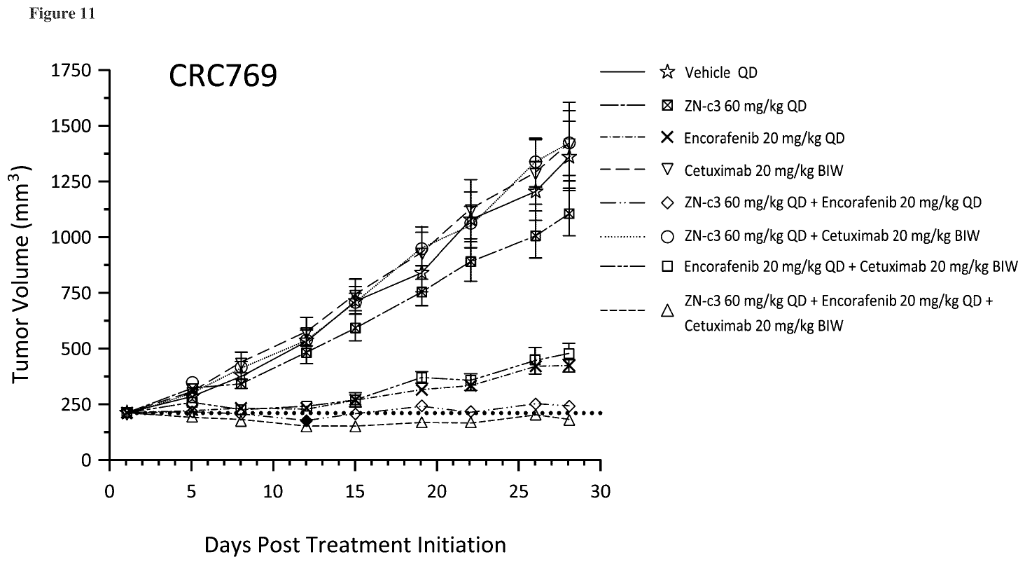

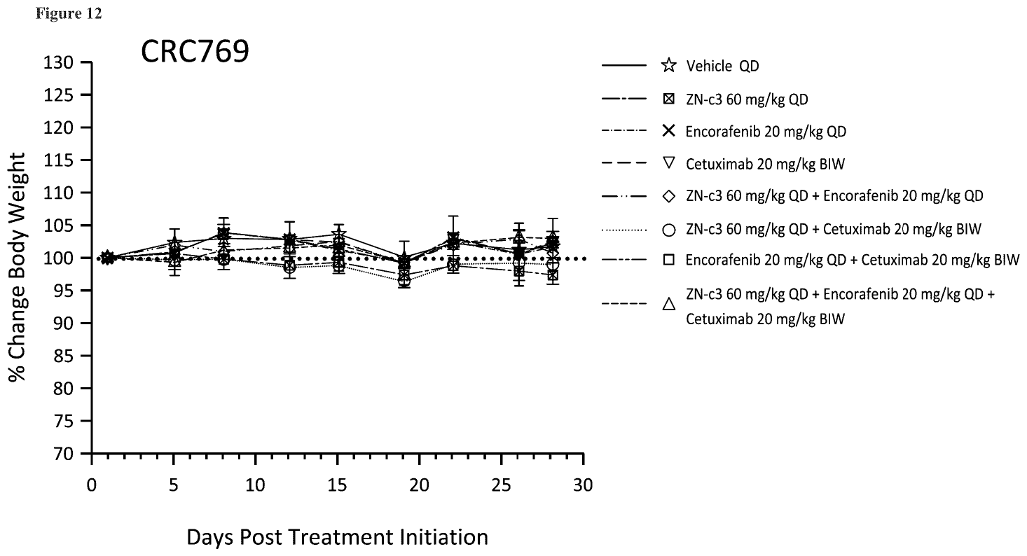

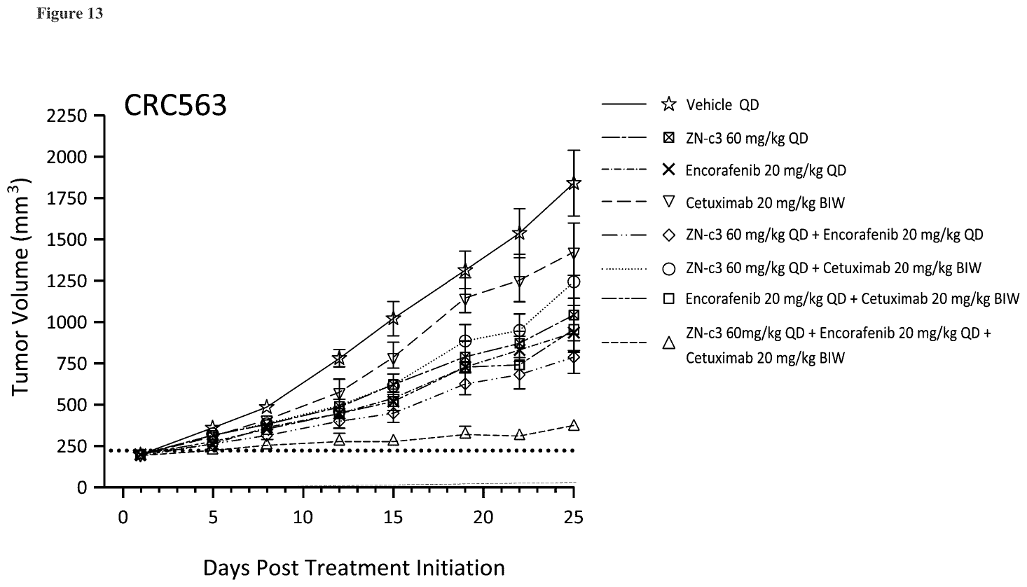

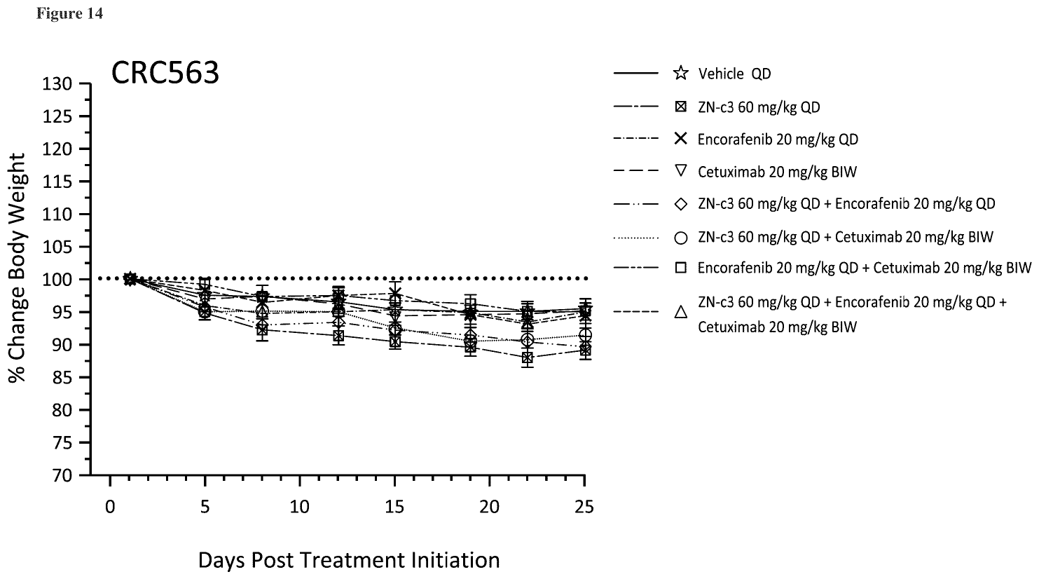

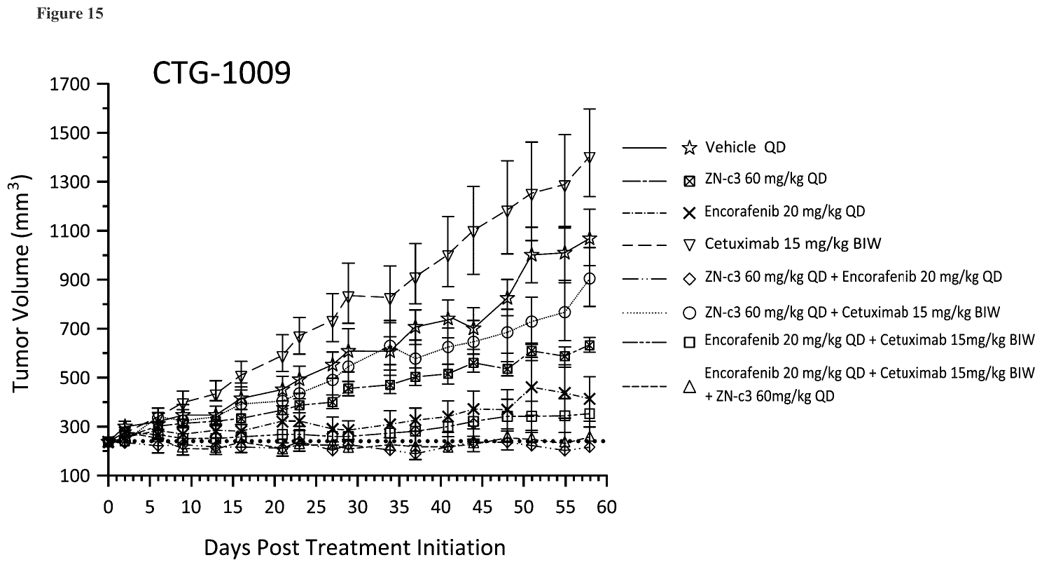

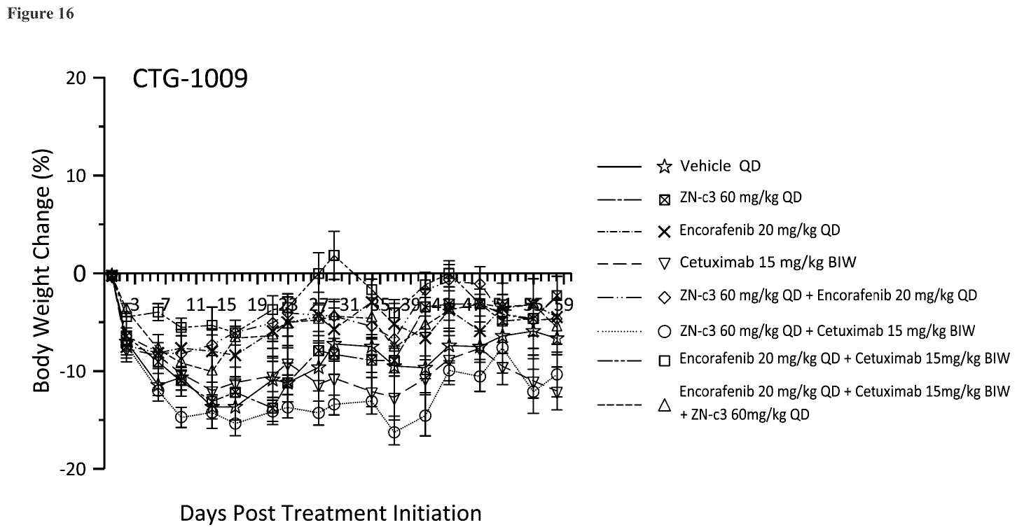

The tumor volume measurements (8 measurements altogether throughout the 28-day studies) for the PDX models are provided in FIG. 11 (CRC769 which is BRAF^{mt} CRC), FIG. 13 (CRC563 which is also BRAF^{mt} CRC), and FIG. 15 (CTG-1009 which is also BRAF^{mt} CRC). Based on the tumor volume measurements, tumor growth inhibition (TGI) values were calculated and are provided in Table 5. In all three PDX models, the triplet therapy of ZN-c3+encorafenib+cetuximab induced tumor regressions that were superior to the current metastatic colorectal cancer standard-of-care double therapy of encorafenib+cetuximab, with TGI values of 86.8 (ZN-c3+encorafenib+cetuximab) as compared to 65.0 (encorafenib+cetuximab) in CRC769; 79.6 (ZN-c3+encorafenib+cetuximab) as compared to 48.7 (encorafenib+cetuximab) in CRC563; and 97.9 (ZN-c3+encorafenib+cetuximab) as compared to 86.9 (encorafenib+cetuximab) in CTG-1009. Moreover, in all three PDX models, the doublet therapy of ZN-c3+encorafenib resulted in higher TGI values than the standard-of-care double therapy of encorafenib +cetuximab, with TGI values of 82.3 (CRC769), 57.2 (CRC563) and 103.44 (CTG-1009) compared to 65.0 (CRC769), 48.7 (CRC563) and 86.9 (CTG-1009). The body weight measurements (8 measurements altogether throughout the 28-day studies) for the PDX models, as shown in FIG. 12 (CRC769), 14 (CRC563), and 16 (CTG-1009) as well as Table 5, indicated minimal body weight changes throughout the studies for the exemplary combination therapies, i.e., triplet therapy of ZN-c3+encorafenib+cetuximab and doublet therapy of encorafenib+cetuximab.

To summarize, the in vivo studies, based on statistical analysis between the standard-of-care doublet therapy (encorafenib+cetuximab) and the triplet therapy (ZN-c3+encorafenib+cetuximab), all CDX and PDX models had statistically significant improvement for the triplet therapy over the doublet therapy with no tolerability issues. Analysis between the standard-of-care doublet therapy (encorafenib+cetuximab) and the doublet therapy of ZN-c3 encorafenib showed that all models, except HT-29, had statistically significant improvement for the latter over the former with no tolerability issues.

TABLE 4

| | | | | Body | Tumor |
|---|---|---|---|---|---|
| | | | | Weight | Vol. |
| | | | | Change | Change |
| | | | | at End of | from D |
| Cell | | | | Treatment | 14-D 21 |
| Line | Compound | Dose | TGI | (%) | (%) |
| HT-29 | Encorafenib | 15 mg/kg QD | 42.6 | 1 | 36.9 |
| | Cetuximab | 15 mg/kg BIW | 12.2 | 7.5 | 38.7 |
| | ZN-c3 | 60 mg/kg QD | 15.0 | 2.3 | 36.3 |
| | ZN-c3 + | 60 mg/kg QD + | 56.4 | −0.4 | 31 |
| | Encorafenib | 15 mg/kg QD | | | |
| | ZN-c3 + | 60 mg/kg QD + | 21.0 | 2 | 37 |
| | Cetuximab | 15 mg/kg BIW | | | |
| | Cetuximab + | 15 mg/kg BIW + | 88.2 | 6.7 | 25.7 |
| | Encorafenib | 15 mg/kg QD | | | |
| | ZN-c3 + | 60 mg/kg QD + | 100.9 | −0.2 | 12.5 |
| | Encorafenib + | 15 mg/kg QD + | | | |
| | Cetuximab | 15 mg/kg BIW | | | |
| LS411N | Encorafenib | 20 mg/kg QD | 64.6 | 0 | 36.6 |
| | Cetuximab | 15 mg/kg BIW | 15.9 | 2.3 | 44.9 |
| | ZN-c3 | 60 mg/kg QD | 69.0 | 0.2 | 35.8 |
| | ZN-c3 + | 60 mg/kg QD + | 101.7 | 1.4 | −0.02 |
| | Encorafenib | 20 mg/kg QD | | | |
| | ZN-c3 + | 60 mg/kg QD + | 54.1 | −1.5 | 36.5 |
| | Cetuximab | 15 mg/kg BIW | | | |
| | Cetuximab + | 15 mg/kg BIW + | 63.4 | −5.3 | 35.1 |
| | Encorafenib | 20 mg/kg QD | | | |
| | ZN-c3 + | 60 mg/kg QD + | 107.6 | 3.1 | −0.16 |
| | Encorafenib + | 20 mg/kg QD + | | | |
| | Cetuximab | 15 mg/kg BIW | | | |

TABLE 5

| | | | | Body Weight |
|---|---|---|---|---|
| | | | | Change at |
| PDX | | | | End of |
| Model | Compound | Dose | TGI | Treatment (%) |
| CRC769 | Encorafenib | 20 mg/kg QD | 69.0 | 1 |
| | Cetuximab | 20 mg/kg BIW | −4.5 | 7.5 |
| | ZN-c3 | 60 mg/kg QD | 19.0 | 2.3 |
| | ZN-c3 + | 60 mg/kg QD + | 82.3 | −0.4 |
| | Encorafenib | 20 mg/kg QD | | |
| | ZN-c3 + | 60 mg/kg QD + | −4.0 | 2 |
| | Cetuximab | 20 mg/kg BIW | | |
| | Cetuximab + | 20 mg/kg BIW + | 65.0 | 6.7 |
| | Encorafenib | 20 mg/kg QD | | |
| | ZN-c3 + | 60 mg/kg QD + | 86.8 | −0.2 |
| | Encorafenib + | 20 mg/kg QD + | | |
| | Cetuximab | 20 mg/kg BIW | | |
| | Cetuximab | 20 mg/kg BIW | 21.8 | −5.2 |
| | ZN-c3 | 60 mg/kg QD | 43.4 | −10.8 |
| | ZN-c3 + | 60 mg/kg QD + | 57.2 | −10.0 |
| | Encorafenib | 20 mg/kg QD | | |
| | ZN-c3 + | 60 mg/kg QD + | 32.3 | −8.5 |
| | Cetuximab | 20 mg/kg BIW | | |
| | Cetuximab + | 20 mg/kg BIW + | 48.7 | −4.8 |
| | Encorafenib | 20 mg/kg QD | | |
| | ZN-c3 + | 60 mg/kg QD + | 79.6 | −5.0 |
| | Encorafenib + | 20 mg/kg QD + | | |
| | Cetuximab | 20 mg/kg BIW | | |
| CTG- | Encorafenib | 20 mg/kg QD | 80.0 | −4.4 |
| 1009 | Cetuximab | 15 mg/kg BIW | −42.6 | −13.0 |
| | ZN-c3 | 60 mg/kg QD | 51.2 | −5.5 |
| | ZN-c3 + | 60 mg/kg QD + | 103.44 | −4.9 |
| | Encorafenib | 20 mg/kg QD | | |
| | ZN-c3 + | 60 mg/kg QD + | 19.0 | −12.2 |
| | Cetuximab | 15 mg/kg BIW | | |
| | Cetuximab + | 15 mg/kg BIW + | 86.9 | −2.7 |
| | Encorafenib | 20 mg/kg QD | | |
| | ZN-c3 + | 60 mg/kg QD + | 97.9 | −3.6 |
| | Encorafenib + | 20 mg/kg QD + | | |
| | Cetuximab | 15 mg/kg BIW | | |

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the present disclosure.

What is claimed is:

1. Use of a combination of compounds for treating a disease or condition, wherein the combination comprises an effective amount of Compound (A), or a pharmaceutically acceptable salt thereof, and an effective amount of Compound (B), or a pharmaceutically acceptable salt thereof; wherein Compound (A) is 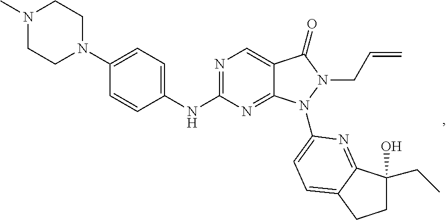 [SMILES: C=CCn1c(=O)c2cnc(Nc3ccc(N4CCN(C)CC4)cc3)nc2n1-c1ccc2c(n1)[C@@](O)(CC)CC2]
or a pharmaceutically acceptable salt thereof; and Compound (B) is a BRAF inhibitor, or a pharmaceutically acceptable salt thereof.

2. The use of claim 1, wherein the BRAF inhibitor is selected from the group consisting of vemurafenib, dabrafenib, encorafenib, agerafenib, AZ-628, belvarafenib, BMS-908662, CHIR-265, DP-4978, GDC-0879, GW5074, lifirafenib, SB590885, naporafenib, PLX-4720, PLX-8394, ABM-1310, ASN-003, JZP815 KIN-2787, and a pharmaceutically acceptable salt of any of the foregoing.

3. The use of claim 2, wherein the BRAF inhibitor is vemurafenib, or a pharmaceutically acceptable salt thereof.

4. The use of claim 2, wherein the BRAF inhibitor is dabrafenib, or a pharmaceutically acceptable salt thereof.

5. The use of claim 2, wherein the BRAF inhibitor is encorafenib, or a pharmaceutically acceptable salt thereof.

6. The use of any one of claims 1-5, wherein the combination further comprises an effective amount of Compound (C), or a pharmaceutically acceptable salt thereof, wherein Compound (C) is an EGFR inhibitor, or a pharmaceutically acceptable salt thereof.

7. The use of claim 6, wherein the EGFR inhibitor is a tyrosine kinase inhibitor.

8. The use of claim 6, wherein the EGFR inhibitor is a monoclonal antibody or an antigen-binding fragment thereof.

9. The use of claim 6, wherein the EGFR inhibitor is selected from the group consisting of afatinib, dacomitinib, erlotinib, gefitinib, osimertinib, cetuximab, necitumumab, nimotuzumab, panitumumab and N-(5-((4-(1-(bicyclo[1.1.1]pentan-1-yl)-1H-indol-3-yl)pyrimidin-2-yl)amino)-2-((2-(dimethylamino)ethyl)(methyl)amino)-4- methoxyphenyl)acrylamide, and a pharmaceutically acceptable salt or an antigen-binding fragment of any of the foregoing.

10. The use of any one of claims 1-9, wherein the disease or condition is colorectal cancer.
1. The use of claim 10, wherein the colorectal cancer is advanced colorectal cancer.
2. The use of claim 10, wherein the colorectal cancer is metastatic colorectal cancer.

11. The use of claim 11 or claim 12, wherein the advanced and/or the metastatic colorectal cancer has progressed following 1 or 2 prior treatment regimens.

12. The use of any one of claims 10-13, wherein the colorectal cancer has a BRAF mutation.

13. The use of any one of claims 10-14, wherein the BRAF mutation is an activating mutation.

14. The use of claim 14, wherein the BRAF mutation occurs at the V600 codon.

15. The use of claim 16, wherein the BRAF mutation is V600E.

18. The use of any one of claims 1-17, wherein the disease or condition is BRAF V600E-mutant metastatic colorectal cancer.
